(19) United States
(12) Patent Application Publication
(10) Pub. No.: US 2007/0032731 A1
(43) Pub. Date: Feb. 8, 2007

(54) NON-INVASIVE PULSE RATE DETECTION VIA HEADPHONE MOUNTED ELECTRODES / MONITORING SYSTEM

(76) Inventors: Jeffrey L. Lovejoy, Laguna Niguel, CA (US); Joseph D. Giordano, Henderson, NV (US)

Correspondence Address: LEWIS AND ROCA LLP, 40 NORTH CENTRAL AVENUE, PHOENIX, AZ, 85004, US

(21) Appl. No.: 11/462,675

(22) Filed: Aug. 4, 2006

Related U.S. Application Data
(60) Provisional application No. 60/705,976, filed on Aug. 5, 2005.

(51) Int. Cl. A61B 5/02

(52) U.S. Cl. 600/500; 600/502; 128/905

(57) ABSTRACT

One or more car phone speakers having functionality to detect heart beats proximal a wearer's respective ears generate electronic signals representing the heart beat over a time interval to derive there from a pulse rate. An audio rendering of the derived pulse rate is made at one or more the ear phone speakers. Heart beat can be combined with other data to produce other such audio renderings.

### RELATED APPLICATIONS

This application claims priority to U.S. Provisional Application Ser. No. 60/705,976, filed on Aug. 5, 2005, titled “Non-Invasive Pulse Rate Detection Via Headphone Mounted Electrodes/Monitoring System”, which is incorporated herein by reference.

### TECHNICAL FIELD

This invention relates to pulse rate detection, and is more particularly related to a method, apparatus, and system to non-invasively detect the pulse rate of a person and to render the detected pulse rate to an earpiece speaker worn by the person.

### BACKGROUND

An athlete can monitor their heart beat during exercise. This can be done by touching the skin to feel the pulsatile motion representing a beat of the heart. The heart beats over a time interval are counted to derive pulse rate. For instance, counting the number of heart beats in a six (6) second interval and multiplying by ten (10) will yield pulses per minute. Numerous motivations exist for an athlete to be aware of their pulse rate during exercise. It is generally understood that an athlete's knowledge of their pulse rate during a work out or competition can be a valuable assessment as to the athlete's present well being and performance.

Mechanical and electromechanical pulse rate detection devices, also known as heart rate monitors, are regularly used by athletes to monitor their heart rate while exercising and resting. These devices typically require the athlete to observe a dial, gauge, or readout to see their pulse rate estimated by the device. Typical heart rate monitors consist of two elements, a chest strap and a wrist receiver (which usually doubles as a watch). In use, the athlete must look at the wrist receiver in order to get notice of their pulse rate.

Advanced heart rate models additionally measure heart rate variability to assess a user's fitness. The chest strap has electrodes in contact with the skin to monitor the electrical voltages in the heart as is known in the electrocardiography arts. When a heart beat is detected a radio signal is sent out which the receiver uses to determine the current heart rate. Some heart rate monitors send coded signals from the chest strap to prevent a user's wrist receiver from receiving signals from other nearby exercisers.

There are a wide number of receiver designs, with all sorts of features. These include average heart rate over exercise period, time in a specific heart rate zone, calories burned, and detailed logging that can be stored for future download and further use.

Any change of the athlete's visual focus away from activities at hand during a work out or competition can cause difficulties ranging from mere inconvenience to diminished athletic performance. In would be an advantage in the art to produce a method, apparatus, and system to give an athlete notice of their pulse rate, and other biological information, without requiring a change of the athlete's visual focus.

### SUMMARY

Implementations provide for one or more ear phone speakers having functionality to detect heart beats proximal a wearer's respective ears. Electronic signals representing the heart beat are accounted for over a time interval to derive there from a pulse rate. An audio rendering of the derived pulse rate is made at one or more of the ear phone speakers. Heart beat can be combined with other data to produce other such audio renderings.

### BRIEF DESCRIPTION OF THE DRAWINGS

A more complete understanding of the implementations may be had by reference to the following detailed description when taken in conjunction with the accompanying drawings wherein:

FIG. 1 depicts an exemplary environment for an athlete wearing a portable or handheld computing device capable of audio renderings, such as a digital audio player, where the audio renderings include that of a pulse rate representation detected by and rendered to an ear bud speaker device worn by the athlete, and where the audio renderings are produced by a combination of hardware and software applications;

FIGS. 2-3 depicts an exemplary implementation of complementary electrical schematics providing functionality for the detection heart beats at the left and right ears of a headphone wearer in which are inserted respective heart beat sensors;

FIG. 4 depicts an exemplary process to detect heart beats over a time interval at the ears of a headphone wearer from which audio renderings of a pulse rate derived there from are made through the headphone, as well as other audio renderings.

### DETAILED DESCRIPTION

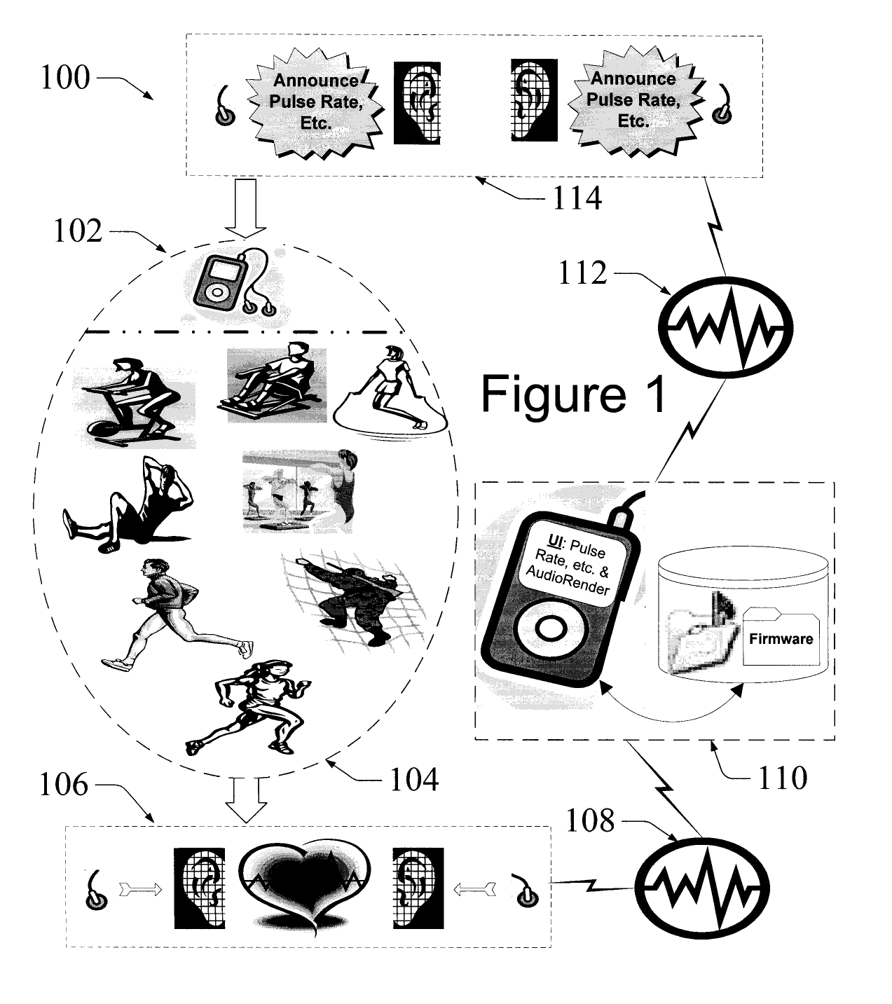

FIG. 1 depicts an environment 100 in which a digital audio player 102 is used by an athlete 104. A digital audio player is (DAP) is a device that stores, organizes and plays digital music files. Though DAPs are typically referred to as MP3 player due to the ubiquity of digital music files in that particular format, DAPs often play many additional file formats. Some such formats are Windows Media Audio (WMA) and Advance Audio Codec (AAC). Commerically popular brands of DAPs include the iPod™ from the Apple Corporation, the iAudio™ from the Cowon Systems, Inc., the Dell Digital Jukebox™ (“Dell DJ”) series from the Dell Corporation, the Creative Nomad/Creative Zen line of digital audio players from the Creative Labs company, the iRiver™ players by the iRiver, the Rio Audio from Digital Networks North America, Inc. (DNNA), the Gmini400 from the Archos company, the GigaBeat™ from the Toshiba Corporation, the mirobe™ from the Olympus company, the Yepp™ from the Samsung company, and the Network Walkman™ from the Sony corporation. Of course, other handheld computing devices can also render audio, including cellular telephones, Personal Digital Assistants (PDA), and other such devices having an operating system (O/S) such as the PALM™ O/S or the POCKET PC™ O/S.

A portable audio device typically is used with headphones to which audio is rendered for the listening pleasure of the wearer. Headphones (also known as earphones, stereo phones, headsets, or the slang term ‘cans’) are a pair of transducers that receive an electrical signal from a media player or receiver and use speakers placed in close proximity to the ears (hence the name earphone) to convert the signal into audible sound waves. They are normally detachable, using a jack plug. Typical products to which they are attached include the ‘walkman’, cellular telephone, CD player, DAP, and PDA. Some headphone units are self-contained, incorporating a radio receiver. Other headphones are cordless, using radio (for example analogue FM, digital blue tooth, Wi-Fi) or infrared signals to communicate with a “base” unit.

Headphones may be used to prevent other people from hearing the sound either for privacy or to protect others. They are also used to exclude external sounds, particularly in sound recording studios and in noisy environments. Headphones generally use a 3.5 mm “mini pin” jack.

Some headphones are worn over the ear. Others are worn within the ear, such as ear buds and canalphones. Ear buds, also know as earphones in British English, are small headphones that are placed directly outside of the ear canal, but without fully enveloping it. Ear buds are generally inexpensive and are favored for their portability and convenience. However, due to their inability to provide isolation, they are not capable of delivering the precision and range of sound offered by many full-sized headphones and canalphones. Ear buds are typically bundled with personal stereos in consumer electronics purchases. For example, the distinctive white headphones that are included with the iPod are ear buds.

Canalphones, also known as “in-ear headphones”, are designed to be placed inside the ear canal, positioning them closer to the eardrum than other types of headphones. They provide better isolation quality (up to 25 dbs) than ear buds because they fit in much the same way as earplugs. Acoustic isolation from canalphones is generally superior to that provided by active noise cancellation mechanisms. Hearing aids are a type of canalphone. Canalphones are traditionally used by live performers as an alternative way of monitoring their music as they allow the performer to protect themselves from the high amount of competitive stage noise present, while maintaining audio fidelity. Also, as canalphones can be molded in various colors and sizes, a flesh tone that completely fits inside the ear is commonly preferred by performers for its discreetness. Canalphone manufacturers include the Shure company, the Sony Corporation, the Etymotic Research company, the Sensaphonics company, and Future Sonics Incorporated.

FIG. 1 shows DAP 102 having headphones of the earbud and/or canalphone variety. While DAP 102 is shown as being hardwired to the headphone, it is contemplated that the DAP102 can also be in wireless communication with the headphone radio or infrared signals.

The headphones are preferably modified to include functionality 106 to detect a heartbeat of the athelete 104. The functionality 106 modification produces a signal 108 representing the heart beat of the wearer. Heart beat signal 108 is communicated to the DAP 102 having an internal archetecture 110 that includes hardware, firmware, software, or combinations thereof. By way of example, and not by way of limitation, internal archetecture 110 has an O/S that works with a file system. The file system includes folders for digital music files that, when rendered into signals 112, produce aubile sounds 114 through the headphones. Other folders include firmware for providing a User Interface (UI) at a display on the DMP 102. The UI allows the wearer to input data into DMP 102, such as a demand request for a song in the digital music file folder to be rendered to the headphone. The UI also allows the wearer to input a demand request to initate a software routine that, working with one of more sensors in the headphones, detects heart beats and derives therefrom a pulse rate. Other applications can be selected by the user with the UI for the detection of other biological information, as explained herein. A software routine then renders an audible report 114 of the requested biological information (e.g., the derived pulse rate) to the headphones as shown in FIG. 1.

The UI can be configured to prompt the wearer for other data, such as birth date or age, weight, level of exercise intensity (i.e., low, moderate, intense), a selection as to a particular type of exercise that is or is to be undertaken (e.g., bicycle riding, walking, jogging, running, weight lifting, callastinics, jumping rope, rowing, obstacle course navigation, resting, etc.). For these data, computations can be made, in combination with the derivation of pulse rate, for still further audible renderings at one or more of the headphones.

As a matter of acoustics esthetics, internal archetecture 110 may include an application that, when executed, periodically lowers and raises the volume of audio renderings in one or more of the earpieces. When the sound volume has been lowered, an audio rendering of the wearer's pulse rate (and other such aduible informational renderings) will be made at substantially full volume so as to best enable the wearer periodic notice of the information they have requested via the UI of the DMP 102. As such, environment 100 shows a loop executing between reference numerals 102-114.

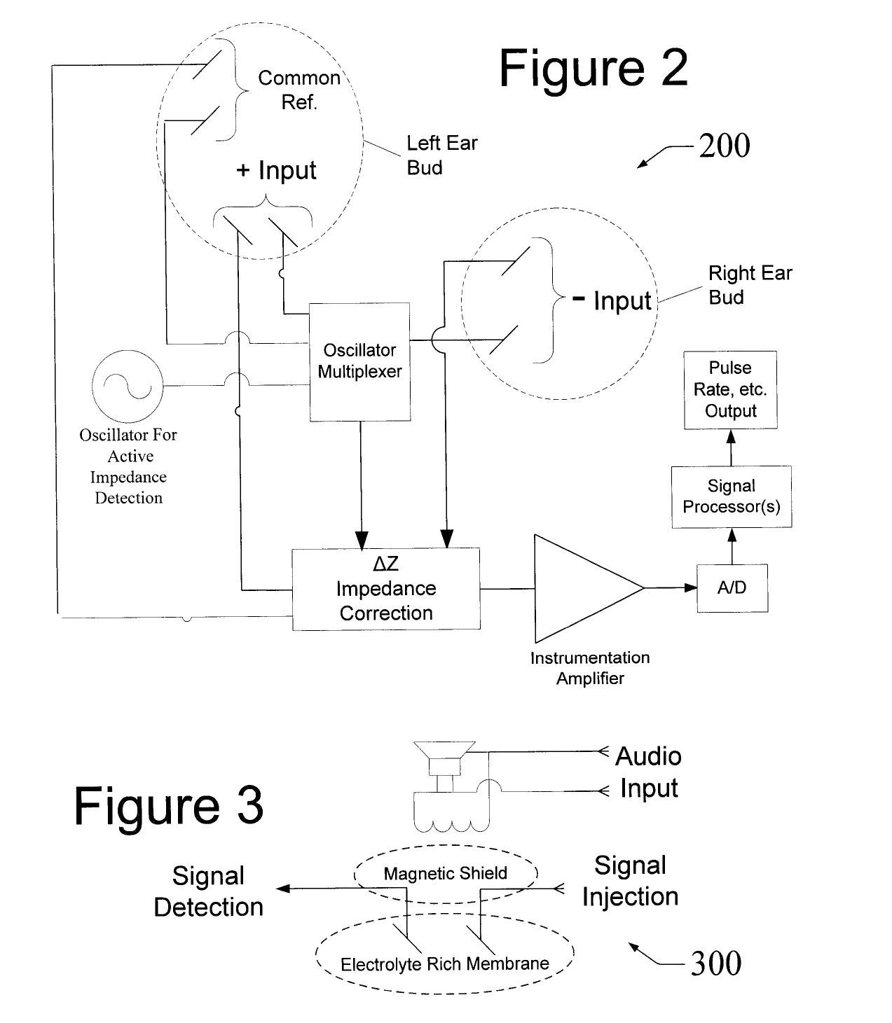

FIGS. 2-3 depicts an exemplary implementation of complementary electrical schematics providing functionality for the detection heart beats at the left and right ears of a headphone wearer in which are inserted respective heart beat sensors. The function of the circuitry in FIGS. 2-3 is, in part, to be an active impedance detection and correction circuit so that there will be an improved common mode noise reduction. These schematics can be used in conjunction with QRS complex detection. The QRS complex is the principal deflection of an electrocardiogram (ECG) that is produced by depolarization of the ventricles (e.g., that part of an ECG rhythm showing electrical activity in the ventricle muscle). The electrical activity in the ventricle muscle, detected non-invasively via sensors in the ear bud headphones, can then be used to determine heart beats per minute (e.g., pulse rate). The QRS complex detection, when used with the schematics seen in FIGS. 2-3, presents an active impedance detection with correction and noise reduction.

Relative to common mode noise reduction, EKG machines inject a weak signal through the electrodes and measure it on an opposite side to make sure there is signal continuity in order to detect whether an electrical lead has been taken off the skin of a patient that is being monitored. Another step is taken by generating different frequencies, or one frequency that will come back as different amplitudes, given that the impedance on each leg of each electrode is known, which here is the plus or minus electrodes and the common reference electrode. Then, an impedance correction can be made with a differential amplifier and electrodes for any mismatch in impedance. The common mode signal, which is to be removed, will be attenuated more on one side than on the opposite side. Thus, the common noise reduction that is desired be removed.

The electrode interface depicted in FIGS. 2-3 is designed for reliable pulse detection. The interface has a tactically soft membrane that fits conformably against sensitive skin tissue in the ear. The membrane is embedded with electrolyte properties so as to be substantially conductive. Because impedance will differ between the ears of a wearer, there can be matching corrections for impedance so as to filter out common mode signals—such as electrical ‘noise’ from muscle artifacts and other artifacts. The signal being detected is similar to a normal EKG lead ‘number one’, which is the lead across the left and right shoulders, though somewhat diminished as it is located further up the wear's body in a narrower vector with less amplitude than a normal EKG. Nevertheless, the R-Wave of the QRS complex of ventricular contraction will thus be detected and used to derive heart rate.

FIG. 3 shows a diagram of a magnetic shield in combination with an electrolyte rich membrane. The electrolyte, for instance, can be supplied and/or supplemented by the wearer in the form of perspiration fluid (e.g., sweat) and the membrane will preferably be porous so as to be in fluid communication with sweat from the skin. The magnetic shield is used to avoid interference from the magnets in the headphones, the magnetic field from which would otherwise interfere with the EKG signal being detected in the ear.

Dashed circles in FIG. 2 each surround two (2) terminals respectively labeled as “− input” and “+ input”. The plus and the minus signs signify inputs to the instrumentation amplifier seen in FIG. 2, which is a differential amplifier to amplify the difference between the plus and the minus inputs. That which is common to both the plus and the minus inputs is subtracted out (e.g., the artifacts of ‘noise’).

Although FIG. 2 shows a minus input indicated for the right ear and a plus input indicated for the left ear, the left and right could be reversed to produce an inverted R-Wave. For the depicted left ear, which is circled with the positive input, there are two prongs labeled “common reference”. The common reference is the reference point for the instrumentation amplifier. In a normal EKG, the Electrical Lead No. 1 would be one plus electrode or one minus electrode on the left shoulder, one on the right shoulder, and a common electrode anywhere else on the body—preferably at the bottom left side of the chest—and used as an offset reference. Thus, ‘noise’ from the body is accounted for and canceled via use of the common reference.

The four prongs circled in FIG. 2, and identified as “ear left”, show two prongs going into an oscillator multiplexor (MUX) to check the impedance from left to right, right to left, common to left, common to right, etc. The two other prongs coming out of the left ear go to a ΔZ Impedance Correction element. Impedance correction can be done in the circuitry as shown, or can be corrected digitally (e.g., via software). Signals produced from the ΔZ Impedance Correction element are routed to the depicted instrumentation amplifier, and then to an analog-to-digital converter, then to a signal processor for digital filtering of the signals so as to output a digital representation of a pulse rate, although the output could alternatively be converted to an analog form. Thus, there is one signal path that comes out of the oscillator multiplexor and goes to an oscillator for active impedance detection. Digital signals out of the oscillator multiplexor are the return signals from the ΔZ Impedance Correction element.

The digital signal from the signal processor is the frequency of the R-Wave. Table lookups, equations, and calculations that use the frequency can derive a numerical equivalent of a pulse rate. The numerical equivalent can then be used to perform an audio rendering at the left and/or right earphones as shown at reference numeral 114 in FIG. 1.

FIG. 3 has an oval the labeled “magnetic shield” that encloses two prongs. These two prongs correspond to the two prongs in FIG. 2 in the dashed ovals labeled “left ear bud” and “right ear bud”. Each one of the prongs in the left ear and in the right ear is encased in a magnetic shield. A coil and speaker are seen in FIG. 3 and represent a headphone. The discrete elements making up the audio input seen in FIG. 3 will preferably be located in the headphone.

Two lines are seen in FIG. 3, one going to an input called “Signal Injection” and the other going to an output called “Signal Detection”. In reference to FIG. 2, the Oscillator for Active Impedance Detection corresponds to the Signal Injection, and the line for Signal Detection routes to the Oscillator Multiplexor. Stated otherwise, the Signal Injection is the output from the Oscillator for Active Impedance Detection, and the Signal Detection is output to the oscillator multiplexor.

In addition to the magnetic shield that surrounds a portion of the two terminals seen in FIG. 3, another dashed oval, labeled “Electrolyte Rich Membrane”, surrounds the two terminals of the signal injection and the signal detection. This membrane is to make contact with both the skin of the wearer and the electrodes. The membrane, which serves as an interface between the skin and the electrode, will preferably be a good conductor of electricity. The two terminals (i.e., electrodes) interfacing with the membrane correspond to electrodes for the left ear and the right ear seen in FIG. 2. Thus, in the left ear and in the right ear of the wearer, there will be both a magnetic shield and an electrolyte rich membrane. The magnetic shield and the electrolyte membrane will preferably both be found in the inner ear canal of the wearer (e.g., incorporated into the ear bud). Audio renderings are made using a discrete element such as a voice coil, as shown in FIG. 3.

In alternative implementations, the sensors in the ear bud will be similar in materials and construction to that of an EKG lead. Alternatively, all or some of the discrete elements depicted in FIG. 2-3 can be replaced by general purpose circuitry executing software to digitally emulate these discrete elements. Still further, one or more Application Specific Integrated Circuits (ASIC) can be used in place of all or some of the discrete elements depicted in FIG. 2-3. Thus, ear buds worn by an athlete can be used to sense heart beats, where those sensed heart beats are used to compute heart rate, and then an audio rendering can be made to inform the athlete of the heart rate that was derived from the sensed heart beats.

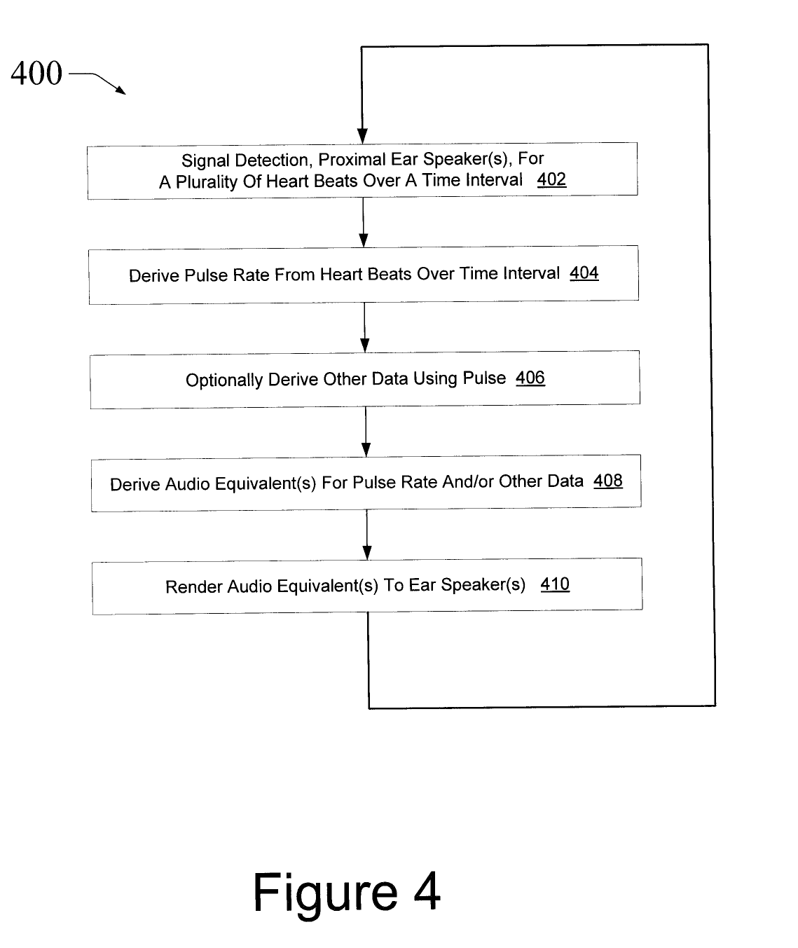

FIG. 4 depicts an exemplary process to detect heart beats over a time interval at the ears of a headphone wearer from which audio renderings of a pulse rate derived there from are made through the headphone, as well as other audio renderings.

At step 402 of process 400, a heat beat of a wearer is detected by a sensor. The sensor creates a signal representing the heart beat. Signals from the sensors are detected over a time interval. One such sensor can be in each earpiece of the wearer's headphones. The sensor can be of a variety that uses electrical conductivity to detect heart beats, as presented with respect to implementations discussed above relative to FIGS. 2-3. Alternatively, one or more emitters can be used to non-invasively irradiate ear tissue with invisible light. One of more detectors can then receive the invisible light that passes through the irradiated ear tissue. Given the invisible light that was emitted by the one or more emitters, and the invisible light that was detected by the one or more detectors, computations can be made with allowances for pulsatile motion of blood within the ear tissue to detect a heart beat as well as biological constituents in the blood (e.g., oxygen content). By way of example, and not by way of limitation, such calculations can make use of the Beer Lambert Law.

In optics, the Beer-Lambert law, also known as Beer's law or the Beer-Lambert-Bouguer law, is an empirical relationship in relating the absorption of light to the properties of the material the light is travelling through. Basically, the law states that absorbance is proportional to the concentration of light-absorbing molecules in the sample. Relavant equations include:

$$A = ε lc , \\ \frac{{I}_{1}}{{I}_{0}} = {ⅇ}^{- α cl} , \\ A = - log \frac{{I}_{1}}{{I}_{0}} , \\ α = \frac{4 π k}{λ} .$$

In the above equations, A is absorbance; C is molar absorptivity; I₀ is the intensity of the incident light; I₁ is the intensity after passing through the material; l is the distance that the light travels through the material (the path length); c is the concentration of absorbing species in the material; a is the absorption coefficient of the absorber; λ is the wavelength of the light; and k is the extinction coefficient:

$${I}_{0} ⩵ > \begin{matrix}\begin{matrix}\begin{matrix}\vert \\ \vert\end{matrix} \\ \vert\end{matrix} \\ <\end{matrix} \begin{matrix}\begin{matrix}\begin{matrix}\cdot \\ \cdot \end{matrix} \\ \cdot \end{matrix} \\ -\end{matrix} \begin{matrix}\begin{matrix}\begin{matrix}\cdot \\ \cdot \end{matrix} \\ \cdot \end{matrix} \\ -\end{matrix} \begin{matrix}\begin{matrix}\begin{matrix}\cdot \\ c , α\end{matrix} \\ \cdot \end{matrix} \\ -\end{matrix} \begin{matrix}\begin{matrix}\begin{matrix}\cdot \\ \cdot \end{matrix} \\ \cdot \end{matrix} \\ 1\end{matrix} \begin{matrix}\begin{matrix}\begin{matrix}\cdot \\ \cdot \end{matrix} \\ \cdot \end{matrix} \\ -\end{matrix} \begin{matrix}\begin{matrix}\begin{matrix}\cdot \\ \cdot \end{matrix} \\ \cdot \end{matrix} \\ -\end{matrix} \begin{matrix}\begin{matrix}\begin{matrix}\vert \\ \vert\end{matrix} \\ \vert\end{matrix} \\ ->\end{matrix} ⩵ > {I}_{1}$$

In essence, the law states that there is an exponential dependence between the transmission of light through a substance and the concentration of the substance, and also between the transmission and the length of material that the light travels through. Thus if l and a are known, the concentration of a substance can be deduced from the amount of light transmitted by it.

The units of c and a depend on the way that the concentration of the absorber is being expressed. If the material is a liquid, it is usual to express the absorber concentration c as a mole fraction (i.e., a dimensionless fraction). The units of a are thus reciprocal length (e.g. cm−1). The law's link between concentration and light absorption is the basis behind the use of spectroscopy.

By way of example, and not by way of limitation, of the use of light to detect heart beat and other biological parameters of the circulatory system, Steuer, et al. disclose a “method and apparatus for non-invasive blood constituent monitoring” in U.S. Pat. No. 6,873,865, issued on Mar. 29, 2005, which is incorporated herein by reference. Steuer, et al. use a clip assembly that may be attached to ear tissue and includes at least a pair of emitters and a photodiode in appropriate alignment to enable operation in either a transmissive mode or a reflectance mode. At least one predetermined wavelength of light is passed onto or through the ear tissue and attenuation of light at that wavelength is detected. Likewise, the change in blood flow is determined by various techniques including optical, pressure, piezo and strain gage methods. Mathematical manipulation of the detected values compensates for the effects of body tissue and fluid. Biological constituents in the blood (i.e., blood oxygen content) can be derived non-invasively using the methods and systems disclosed by Steuer, et al. As with pulse rate, any such biological constituent can be reported in an audible rendering to the headphones of a DMP as set forth herein.

Any of the foregoing technologies, as well as others known in the art, can be used to detect heart beats over a given time period for a determination of pulse rate. All or part of the processing of electrical signals representing heart beats can be perfomed by one or more software applications executed by a DMP (e.g., DMP 102 seen in FIG. 1). Other known methods of non-invasively finding and audibly reporting pulse rate and other biological informaton are also contemplated for use in the inventive method, apparatus, and system.

At step 404, the signals from one or more of the sensors are used to derive the pulse rate over the time interval. Further derivations can optionally be made at step 406 using the heart beats, the pulse rate, and other input provided by the configuration of the DMP and/or the user of the DMP.

At step 408, the derivations made in step 404 are converted into audio equivalent(s) that are to be rendered in step 410 at the wear's headphones. These conversions can be made by a look up between each derivation and its audio equivalent, as well as other conventional techniques for finding equivalents. Step 410 can further include a process to lower volume of other audio renderings so that the biological informational audio renderings (e.g., pulse rate, etc.) can be readily heard and understood by the wearer of the headphones.

Process 400 loops between steps 402 and 410 to give the wearer periodic notice as to the biological information previously requested by the wearer of the headphones, such as by use of a UI for the DMP 102 seen in FIG. 1.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of detecting a QRS complex of a wearer of one or more ear pieces in a headphone for non-invasive pulse rate determination.

2. A method for active impedance detection and correction for common mode noise reduction in heart beat signals sensed via one or more ear pieces in a headphone for non-invasive pulse rate determination.

3. A monitoring system for determination and audible updates reporting of information via headphone mounted sensors.

4. The monitoring system as defined in claim 3, wherein:
the information in the audible updates is reported via the headphone using active impedance detection and correction for common mode noise reduction in signals output by one or more sensors; and
the headphone includes one or more ear pieces.
